(19) United States
(12) Patent Application Publication
(10) Pub. No.: US 2025/0375218 A1
(43) Pub. Date: Dec. 11, 2025

(54) SYSTEMS AND METHODS FOR DELIVERY OF A SUBSTERNAL LEAD OF AN IMPLANTABLE DEVICE

(71) Applicant: North American EP Technology LLC, Edina, MN (US)

(72) Inventors: Robert Joseph GASKILL, Edina, MN (US); Yanhui LI, Edina, MN (US)

(73) Assignee: North American EP Technology LLC, Edina, MN (US)

(21) Appl. No.: 19/300,138

(22) Filed: Aug. 14, 2025

Related U.S. Application Data
(63) Continuation-in-part of application No. PCT/US2025/013826, filed on Jan. 30, 2025.
(60) Provisional application No. 63/627,448, filed on Jan. 31, 2024.

(51) Int. Cl. A61B 17/34; A61N 1/05; A61N 1/39

(52) U.S. Cl. CPC A61B 17/3468; A61N 1/05; A61N 1/3956

(57) ABSTRACT

A system for delivering a lead an implantable diagnostic/treatment device includes a needle defining a needle lumen and including a biased portion. The biased portion is configured to bias at least a distal end portion of the needle toward a posterior sternal wall when disposed in a body of a patient. The system includes a stylet removably coupled to the needle that is configured to selectively straighten the biased portion of the needle for insertion into the body of the patient. The system includes a guidewire configured to extend through the needle lumen and into the substernal space. The system includes a sheath configured to be advanced over the guidewire to dispose a distal end of the sheath in the substernal space. The sheath defines a sheath lumen allowing the lead to be advanced therethrough to deliver the lead to a substernal space in the body of the patient.

### CROSS-REFERENCE TO RELATED APPLICATIONS

This application is a continuation-in-part of International Patent Application No. PCT/US2025/013826, filed Jan. 30, 2025, entitled “Systems and Methods for Delivery of a Substernal Lead of an Implantable Device,” which claims priority to and benefit of U.S. Provisional Patent Application No. 63/627,448, filed Jan. 31, 2024, entitled “Systems and Methods for Delivery of a Lead of an Implantable Cardioverter Defibrillator,” the disclosure of each of which is incorporated herein by reference in its entirety.

### BACKGROUND

The embodiments described herein relate generally to leads of an implantable device and more particularly, to systems and methods for delivering substernal leads for implantable devices such as implantable diagnostic devices, implantable cardioverter defibrillators, and/or the like.

Some known modalities for monitoring, diagnosis, and/or treating physiological and/or pathophysiological conditions include implanting one or more devices in the body of a patient. Implantable devices are typically connected to one or more leads, which among other things, can provide a way of placing sensors, electrodes, and/or components thereof in desired positions in the body (e.g., remote from the implanted device to which the lead is connected). For example, leads (and/or sensors thereof) are often used to detect or measure certain characteristics associated with a patient. The characteristics and/or data indicative of or associated with the characteristics can be used for monitoring physiologic and/or pathophysiologic functions; diagnosing various diseases or disease states, health events, conditions, and/or injuries of a patient; and/or otherwise collecting health-related data for a patient. Moreover, electrodes or other treatment components of the lead can be used to provide one or more treatments, therapies, etc. (e.g., defibrillation shock therapy, cardiac pacing, and/or the like).

For example, the human heart is a mechanical pump for moving blood through the body and is driven by cardiac electrical activities. It therefore follows that cardiac electrical abnormalities (cardiac electrical signals) can result in abnormalities in the mechanical functioning of the pump, which in turn, may hinder the ability of the heart to move blood through the body and/or may otherwise result in abnormal heart function. Moreover, abnormal heart function such as sudden cardiac arrest, arrhythmias, and/or the like can lead to sudden cardiac death.

In some instances, implantable diagnostic and/or treatment devices can be used to detect, diagnose, and/or treatment abnormal cardiac function. Such devices can include but are not limited to, for example, pacemakers, implantable cardioverter defibrillators (ICD), cardiac resynchronization therapy defibrillators (CRT-D), ventricular assist devices, heart failure diagnostic devices, and/or the like. In some traditional procedures, the leads of some such devices are delivered into the heart transvenously, allowing the leads and/or sensors thereof to receive cardiac electrical and/or mechanical signals. Transvenous delivery of traditional leads, however, can result in lead-related patient complications.

In an effort to mitigate such complications, epicardial, substernal, and/or subcutaneous leads and/or sensing electrodes have been developed that are placed external (or at least partially external) to the heart. Placement of leads external to the heart can increase pacing thresholds compared to intracardiac (e.g., transvenously delivered) leads. These higher pacing thresholds may prohibit or limit the ability of implantable devices to leverage cardiac pacing to treat spontaneous ventricular tachycardia or other cardiac electrical states without triggering a painful, high-energy defibrillation shock that may be considered inappropriate. In addition, challenges remain in the discrimination of true cardiac states due to potential confounding of multiple sources of events that can get classified as abnormal cardiac electrical states, without a corresponding or anticipated abnormal cardiac mechanical state (e.g., a reduction of hemodynamic output). For example, a sensor, electrode, etc. may detect an electrocardiogram (ECG) signal that is associated with or otherwise suggests atrial fibrillation, but a diagnostic and/or treatment device may classify the ECG signal as ventricular tachycardia or an ECG signal that is associated with or otherwise suggest noise may be classified as ventricular fibrillation. These misclassifications can lead to misdiagnosis and/or the delivery of inappropriate treatment. Moreover, existing devices and methods of delivering leads external to the heart may result in tissue damage during implantation and/or may lack the ability to deliver certain leads to a desired position relative to anatomic structures (e.g., the sternum, the heart, etc.).

Thus, there is a need for improved systems and methods for delivering substernal leads that can allow for improved decision-making of implantable diagnostic and/or treatment devices (e.g., through the detection and use of signals from multiple sources data).

### SUMMARY

In some embodiments, a system for delivering a lead an implantable diagnostic/treatment device includes a needle defining a needle lumen and including a biased portion. The biased portion is configured to bias at least a distal end portion of the needle toward a posterior sternal wall when disposed in a body of a patient. The system includes a stylet removably coupled to the needle that is configured to selectively straighten the biased portion of the needle for insertion into the body of the patient. The system includes a guidewire configured to extend through the needle lumen and into the substernal space. The system includes a sheath configured to be advanced over the guidewire to dispose a distal end of the sheath in the substernal space. The sheath defines a sheath lumen allowing the lead to be advanced therethrough to deliver the lead to a substernal space in the body of the patient.

In some embodiments, a system includes an implantable cardioverter defibrillator (ICD) configured to be implanted in a patient. The ICD includes a generator configured to generate treatment energy and a lead configured to deliver the treatment energy from the generator to the heart of the patient. The lead includes at least one sensor. The system further includes a lead delivery system configured to deliver the lead to a predefined location in a substernal space of a patient. The lead delivery system includes a needle defining a needle lumen and including a biased portion, a stylet removably coupled to the needle and configured to selectively straighten the biased portion when coupled to the needle, a guidewire configured to extend through the needle lumen and into the substernal space, and a sheath configured to be advanced over the guidewire to dispose a distal end of the sheath in the substernal space. The sheath includes a sheath lumen configured to accept the lead.

In some embodiments, a method for delivering the lead of an implantable device includes inserting a needle with a stylet disposed in a needle lumen thereof into a patient such that a distal end of the needle is disposed in a substernal space of the patient. The method includes withdrawing the stylet to allow a biased portion of the needle to bend with an angle of the posterior sternal wall. The method includes advancing a guidewire through the needle lumen of the needle such that a distal end portion of the guidewire is disposed in the substernal space. The method includes removing the needle from the guidewire while the guidewire remains in the substernal space. The method includes advancing a sheath over the guidewire such that a distal end portion of the sheath is disposed in the substernal space and the lead is advanced through a sheath lumen of the sheath such that a distal end portion of the lead is disposed in the substernal space. The method includes removing the sheath from the patient while maintaining the distal end portion of the lead remains in the substernal space.

### BRIEF DESCRIPTION OF THE DRAWINGS

FIG. 1 schematically depicts a system including an implantable diagnostic/treatment device and a lead delivery system engaging with a patient, according to an embodiment.

FIGS. 2A-2E schematically depicts a lead delivery system used to deliver a lead of an implantable diagnostic/treatment device such as an ICD into a substernal space of a patient, according to an embodiment.

FIG. 3 schematically depicts an example of an ICD, according to an embodiment, having an ICD generator and an ICD lead suitable for delivery into the substernal space of a patient via any of the lead delivery systems and methods described herein.

FIG. 4 is a flow chart depicting a method for positioning a lead of an ICD in the substernal space of a patient, according to an embodiment.

FIG. 5 depicts a stylet disposed within a needle, according to an embodiment.

FIG. 6 depicts a needle with a stylet disposed within engaging a substernal space of a patient.

FIG. 7 depicts the needle of FIG. 6 with the stylet partially retracted.

FIG. 8 depicts the needle of FIG. 6 with the stylet fully retracted.

FIG. 9 depicts the needle of FIG. 6 with a guidewire disposed within and extending into the substernal space.

FIG. 10 depicts the guidewire of FIG. 9 with the needle retracted.

FIG. 11 depicts a sheath disposed on the guidewire of FIG. 10.

FIG. 12 depicts the sheath of FIG. 11 with the guidewire retracted.

### DETAILED DESCRIPTION

The embodiments described herein relate generally to systems and/or methods for delivering a lead of an implantable device into a patient. In some embodiments, the implantable device may be configured to deliver shock therapy based at least in part on one or more characteristics associated with a heart of a patient. In some embodiments, a lead delivery system can be configured to deliver any suitable number of leads having any suitable shape, size, and/or configuration into, for example, a substernal space or anterior mediastinum of the patient.

In some embodiments, the delivery devices, systems, and/or methods described herein can deliver one or more leads configured to be used with any suitable diagnostic/treatment device and/or system. Examples of such diagnostic/treatment devices in which the leads (i.e., delivered using the embodiments and methods herein) are implemented or used can include but are not limited to an implantable cardiac treatment device (e.g., cardiac therapy device, defibrillator, implantable cardioverter defibrillator (ICD), cardiac resynchronization therapy defibrillator (CRT-D), etc.) configured to deliver treatment (shock therapy) based at least in part on one or more characteristics associated with a heart of a patient. Alternatively, the diagnostic/treatment systems and/or methods described herein can be at least partially implemented in or as an implantable diagnostic device and/or can otherwise include an implantable diagnostic device configured to make diagnostic determinations and/or predictions independent of whether a corresponding treatment is provided. It should be understood that the embodiments and methods described herein can be implemented as a diagnostic system, a treatment system, a combined diagnostic/treatment system, etc.

In some embodiments, an ICD, or more specifically a lead of the ICD, can include and/or can be in communication with any number of sensors configured to detect one or more characteristics associated with the heart. The one or more characteristics can also include characteristics that are not detected by the sensors, such as patient demographic and/or health data (e.g., age, genetic information, health records, etc.). The one or more characteristics can be correlated and used to determine whether to provide treatment. In some embodiments, the ICD lead can include one or more electrode configured to deliver electric energy to the heart of a patient (e.g., at least one electrode configured to deliver relatively high-power energy for shock therapy (defibrillation therapy), at least one electrode configured to deliver relatively low-power energy for anti-tachycardic pacing, and/or any other suitable electrode(s) or combination(s) thereof). The systems and methods described herein are configured to deliver one or more ICD leads into the substernal space or the anterior mediastinum of a patient such that one or more portions of the ICD is/are placed in desired position(s) relative to the heart (e.g., the sensor(s), electrode(s), and/or any other features of the ICD lead are in desired position(s) relative to the heart). In some embodiments, the systems and methods described herein can be configured to deliver and/or position the ICD lead(s) relative to the heart is such a manner that allows the ICD to provide shock therapy (e.g., pacing therapy, defibrillation therapy, etc.) while reducing the delivery of inappropriate or undesired shocks.

In some embodiments, the delivery systems and/or delivery methods described herein can use a needle with a biased portion or a needle that is otherwise reconfigurable to enable placement of the ICD lead in a desirable position in the substernal space and/or anterior mediastinum of the patient. More specifically, the substernal space or anterior mediastinal space is an anatomical position in the body that is inconsistent with an insertion angle of a straight needle or puncture device. Accordingly, to facilitate access of the substernal space (or anterior mediastinum) it may be desirable to use a needle or puncture device that is at least selectively biased or otherwise reconfigurable from, for example, an insertion configuration (e.g., a straight configuration) to a delivered or positioned configuration (e.g., biased into the substernal space and/or toward the posterior sternal wall). In some embodiments, the systems and methods described herein are configured to decrease the likelihood of puncture trauma, contact/puncture of the pericardium, heart, and/or vessels, tissue damage, undesirable ICD lead placement, etc. In some embodiments, the system and methods described herein are configured to place a portion of the ICD lead against the posterior sternal wall. In some embodiments, the systems and methods described herein allow for a portion of the lead to contact at least one of the pericardium or the posterior sternum.

The terminology used herein is for the purpose of describing particular embodiments, implementations, and/or concepts (including any feature(s) or aspect(s) thereof) and is not intended to be limiting. Unless defined otherwise, technical and/or scientific terms used herein have the same meanings as commonly understood by one of ordinary skill in the art. Any explanation or discussion of or using particular terms is intended to provide context and to facilitate understanding and is not necessarily intended to replace or supersede commonly used or known definitions understood by one skilled in the art unless explicitly stated otherwise. Moreover, various terms may be used to describe similar or substantially the same embodiments, implementations, and/or concepts (including any feature(s) or aspect(s) thereof) and thus, the use of particular term is not intended to be limiting and/or to the exclusion of other terms unless the terms are mutually exclusive, or the context clearly states otherwise.

As used herein, the singular forms “a,” “an,” and “the” are intended to include the plural forms as well, unless the context clearly indicates otherwise. With respect to the use of singular and/or plural terms herein, those having skill in the art can translate from the singular to the plurality and/or vice versa as is appropriate for the context and/or application. Furthermore, any reference herein to a singular component, feature, aspect, etc. is not intended to imply the exclusion of more than one such component, feature, aspect, etc. (and/or vice versa) unless expressly stated otherwise. The various singular/plural permutations may be expressly set forth herein for sake of clarity.

In general, terms used herein and in the appended claims are intended as “open” terms unless expressly stated otherwise. For example, the term “including” should be interpreted as “including but not limited to,” the term “having” should be interpreted as “having at least,” etc. Similarly, the term “comprising” may specify the presence of stated features, elements, components, integers (or fractions thereof), steps, operations, and/or the like but does not preclude the presence or addition of one or more other features, elements, components, integers (or fractions thereof), steps, operations, elements, components, and/or groups thereof, and/or the like unless such combinations are otherwise mutually exclusive.

As used herein the term “and/or” includes any and all combinations of one or more of the associated listed items. It should be understood that any suitable disjunctive word and/or phrase presenting two or more alternative terms, whether in the written description or claims, contemplates the possibilities of including one of the terms, either of the terms, or both/all of the terms. For example, the phrase “A and/or B” will be understood to include the possibilities of “A” alone, “B” alone, or a combination of “A and B.”

All ranges described herein include each individual member or value and are intended to encompass any and all possible subranges and/or combinations of subranges thereof unless expressly stated otherwise. Any listed range should be recognized as sufficiently describing and enabling the same range being broken down into at least equal subparts unless expressly stated otherwise.

As used herein, the terms “about,” “approximately,” and/or “substantially” when used in connection with stated value(s) and/or geometric structure(s) or relationship(s) is intended to convey that the value or characteristic so defined is nominally the value stated or characteristic described. In some instances, the terms “about,” “approximately,” and/or “substantially” can generally mean and/or can generally contemplate a value or characteristic stated within a desirable tolerance (e.g., plus or minus 10% of the value or characteristic stated). For example, a value of about 0.01 can include 0.009 and 0.011, a value of about 0.5 can include 0.45 and 0.55, a value of about 10 can include 9 to 11, and a value of about 1000 can include 900 to 1100. Similarly, a first surface may be described as being substantially parallel to a second surface when the surfaces are nominally parallel. While a value, structure, and/or relationship stated may be desirable, it should be understood that some variance may occur as a result of, for example, manufacturing tolerances or other practical considerations (such as, for example, the pressure or force applied through a portion of a device, conduit, lumen, etc.). Accordingly, the terms “about,” “approximately,” and/or “substantially” can be used herein to account for such tolerances and/or considerations.

As used herein, the words “proximal” and “distal” refer to the direction closer to and away from, respectively, a user who would place the device into contact with a patient. The words “proximal” or “distal” can be relative terms and do not necessarily refer to universally fixed positions or directions. Thus, for example, the end or end portion of a device first touching the body of the patient would be the distal end or distal end portion, while the opposite end or end portion of the device (e.g., the end or end portion of the device being manipulated by the user) would be the proximal end or proximal end portion of the device.

As used herein, the term “cardiac signals” generally refers to signals from one or more sensors that can include physiological or pathophysiological bio-signals from the heart. Such signals can be, for example, cardiac electrical signals or cardiac non-electrical signals. Cardiac electrical signals can include any suitable signals associated with and/or otherwise indicative of the electrical functioning of the heart. The measurement of such cardiac electrical signals may include, but is not limited to, heart rate, voltage, P wave, QRS morphology, ST segment, T wave, electrocardiogram (ECG) diagnosis, and/or the like, sensed through any suitable number of vectors. Cardiac non-electrical signals (also referred to herein as “cardiac mechanical signals”) can include any suitable signals associated with and/or otherwise indicative of the non-electrical (e.g., mechanical) functioning of the heart. The measurement of such cardiac mechanical signals may include, but is not limited to, pressure characteristics (e.g., blood pressure, pressure in the tissue or volumes surrounding the heart, venous pressures, arterial pressures, and/or changes in such pressures, etc.), hemodynamic characteristics, oxygen saturation, sensed mechanical heart movement, cardiac sounds, cardiac echogram (ultrasound), cardiac Doppler, and/or the like.

The embodiments described herein and/or portions thereof can be formed or constructed of one or more biocompatible materials. In some embodiments, the biocompatible materials can be selected based on one or more properties of the constituent material such as, for example, stiffness, toughness, durometer, bioreactivity, etc. Examples of suitable biocompatible materials include but are not necessarily limited to metals, glasses, ceramics, and/or polymers. Examples of suitable metals include pharmaceutical grade stainless steel, gold, titanium, nickel, iron, platinum, tin, chromium, copper, and/or alloys thereof. A biocompatible polymer material may be biodegradable or non-biodegradable. Examples of suitable biocompatible polymer materials can include but are not necessarily limited to polylactides, polyglycolides, polylactide-co-glycolides, polyethylene-glycols, polyanhydrides, polyorthoesters, polyetheresters, polycaprolactones, polyesteramides, poly(butyric acid), poly(valeric acid), polyurethanes, polyamides (nylons), polyesters, polycarbonates, polyacrylates, polystyrenes, polypropylenes, polyethylenes, polyethylene oxide, polyolefins, polyethersulphones, polysulphones, polyvinylpyrrolidones, polyvinyl chloride, polyvinyl fluoride, poly(vinyl imidazole), polyether urethanes, silicone polyether urethanes, polyetheretherketones (PEEK), polytetrafluoroethylenes (PTFE), polylactones, chlorosulphonate polyolefins, ethylene-vinyl acetates and other acyl substituted cellulose acetates, elastomers, thermoplastics, and/or blends and copolymers thereof.

The embodiments, methods, and/or implementations herein, and/or the various features or advantageous details thereof, are explained more fully with reference to the non-limiting examples illustrated in the accompanying drawings and detailed in the following description. The examples and/or embodiments described herein are intended to facilitate an understanding of structures, functions, and/or aspects of the embodiments, ways in which the embodiments may be practiced, and/or to further enable those skilled in the art to practice the embodiments herein. Similarly, methods and/or ways of using or implementing the embodiments described herein are provided by way of example only and not limitation. Specific uses and/or implementations described herein are not provided to the exclusion of other uses unless the context expressly states otherwise. For example, while some embodiments herein describe a lead used with, for example, an implantable cardioverter defibrillator, in some implementations, the leads described herein can be used with other suitable treatment devices such as a cardiac therapy device, a CRT-D, an implanted electric stimulator, a pacemaker, etc. In some implementations, the leads described herein can be used with implantable diagnostic devices or combination diagnostic/treatment devices. Accordingly, while examples describe leads used with ICDs, it should be understood that such examples are not provided to the exclusion of other uses (e.g., with diagnostic devices or combined diagnostic/treatment devices) unless the context expressly states otherwise. Descriptions of well-known components, methods, techniques, etc. may be omitted so as to not obscure the embodiments herein. Like numbers refer to like elements throughout.

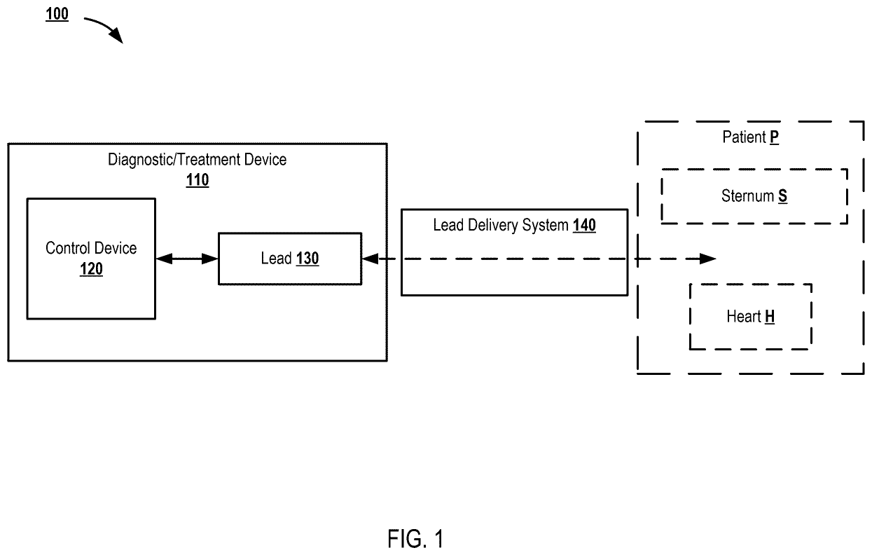

FIG. 1 schematically depicts a system 100 including an diagnostic/treatment device 110 and a lead delivery system 140 configured to deliver one or more portions of the diagnostic/treatment device 110 into a patient P, according to an embodiment. The diagnostic/treatment device 110, or a portion thereof, is implanted (or implantable) in the patient P via the lead delivery system 140. The diagnostic/treatment device 110 can be a device configured to deliver treatment to, monitor, and/or diagnose a patient P based on a position in the in the substernal space. For example, the diagnostic/treatment device 110 can be implemented as an ICD and can be utilized for treating certain conditions or states of a heart H of the patient P via electric treatment therapies after the lead delivery system 140 delivers the one or more portions of the diagnostic/treatment device 110.

As shown in FIG. 1, the diagnostic/treatment device 110 can include control device 120 operatively coupled to an lead 130. The diagnostic/treatment device 110 can be any suitable implantable cardioverter defibrillator device and/or system. In some implementations, for example, the diagnostic/treatment device 110 can be similar to and/or substantially the same as any of the ICDs (or portions thereof) described in U.S. Pat. No. 12,337,184 (“the '184 patent”), filed Dec. 5, 2023, entitled “Systems, Devices, and Methods for Improving Patient Outcomes in Implantable Cardioverter Defibrillators,” International Patent Application No. PCT/US2025/35229 (“the '229 PCT”), filed Jun. 25, 2025, entitled “Systems, Devices, and Methods for Substernal Leads for Applying Cardiac Treatment,” and International Patent Application No. PCT/US2024/042740 (“the '740 PCT”), filed Aug. 16, 2024, entitled “Systems, Devices, and Methods for Improving Decision-Making of Implantable Devices Using Multiple Data Sources,” the disclosures of which are incorporated herein by reference in their entireties. Accordingly, portions and/or aspects of the diagnostic/treatment device 110 may be generally described below for context but are not described in detail with respect to the embodiment shown in FIG. 1.

For example, the control device 120 included in the diagnostic/treatment device 110 can be any suitable device or combination of devices configured to receive signals from the lead 130, monitor the received signals, determine a diagnostic status based, in part, at least on the signals, determine when to provide treatment based at least in part on the signals, and/or generate treatment energy that can be delivered to the heart H via the lead 130. In some embodiments, the control device 120 can be placed on the pectoralis major muscle of the patient P, behind the pectoral major muscle, on the abdomen, along the left exterior thorax, or elsewhere on or in the body of the patient P. Although not shown in FIG. 1, the control device 120 can include a processor, a memory, a power system, a treatment generator, and/or a lead interface, as described, for example, in the '184 patent.

The control device 120 is electrically and/or electronically coupled to the lead 130 allowing signals and/or electric energy to be transferred therebetween. For example, the control device 120 can receive signals from the lead 130 that include data (e.g., sensor data) representing measurements associated with one or more characteristics of the patient P or the heart H. In some embodiments, the control device 120 can include a power system configured to store and/or generate energy for use by the diagnostic/treatment device 110 and to select, determine, and/or define treatments for delivery to the patient P. The power system can also be configured to generate a therapy signal (e.g., anti-tachycardia pacing, defibrillator shock, etc.). For example, the therapy signal can include treatment energy for at least one of anti-tachycardia pacing and/or shock therapy. In some implementations, the treatment energy for anti-tachycardia pacing can be low-power or relatively low-power treatment energy and the treatment energy for shock therapy can be high-power or relatively high-power treatment energy. In some embodiments, the control device 120 is configured to monitor the patient P and/or determine a diagnostic status associated with the patient. In some embodiments, the diagnostic status can be associated with characteristics of the patient being monitored over a period of time. Monitoring and/or diagnosing is/are further described in the '740 PCT.

The lead 130 is coupled and/or electrically connected to the control device 120 (e.g., via a lead interface). In some embodiments, the lead 130 can extend away from the control device 120 via a conduit, connector, tube, shaft, etc., that includes at least one signal/power carrying wire. In some embodiments, the lead 130 is configured to deliver treatment to the patient P and/or the heart H of the patient P. More specifically, in some implementations, the lead 130 is configured to be deployed and/or utilized at least partially outside of the heart H. Similarly stated, the lead 130 is configured for use outside of the chambers of the heart H and, as such, is not a traditional, transvenously delivered ICD lead. For example, the lead 130 can be a pericardial lead, an epicardial lead, and/or the like (or combinations thereof). In some embodiments, the lead 130 is configured to be disposed in the substernal space for monitoring one or more characteristic associated with the patient P.

The lead 130 can include and/or can be in communication with one or more sensors (e.g., via a sensor interface and/or the like) configured to measure a set of health characteristics associated with the heart H of the patient P. In some embodiments, the lead 130 can include and/or can be in communication with multiple sensors, each of which is configured to measure a different health characteristic of the patient P (or at least the heart H of the patient). For example, in some embodiments, the lead 130 can be similar to and/or substantially the same as those described in the '184 patent and/or the '229 PCT. In some embodiments, the lead 130 can include, for example, a first sensor configured to measure cardiac electrical signals (e.g., heart rate, voltage, P wave, QRS morphology, ST segment, T wave, electrocardiogram (ECG) signals, etc.), and a second sensor configured to measure cardiac mechanical signals (e.g., hemodynamic status, hemodynamic output, blood pressure or other pressures within the body, and/or derivative(s) thereof). In some embodiments, the lead 130 can include one or more pressure sensor configured to measure a pressure in the anterior mediastinum.

As described in further detail herein, the lead 130 can be delivered (e.g., via the lead delivery system 140) such that the one or more sensors are positioned in the substernal space, anterior mediastinum, and/or otherwise positioned so that the sensors contact, engage, or are in close proximity to the heart H (e.g., the fibrous pericardium) of the patient P. In some embodiments, one or more sensor(s) can be positioned in any other place in or on the body of the patient P where the sensor(s) can measure the desired cardiac signals (e.g., cardiac electrical signals, cardiac mechanical signals, and/or any other suitable signals). In some embodiments, one or more sensor(s) can be positioned in the epicardium of the heart H.

The lead 130 can include any number of shocking elements, electrodes, conductors, etc. for delivering treatment (e.g., including energy from the control device 120) to the heart H of the patient P. In some embodiments, the lead 130 can include biased portions, loops, coils, waves, and/or any other shape, geometry, and/or feature that can aid in positioning all or a subset of the shocking elements relative to the heart H when the lead 130 is in the substernal space. In some embodiments, the lead 130 can include shocking elements, electrodes, conductors, etc. configured to deliver relatively high energy shock treatment to the heart H (e.g., for ventricular defibrillation treatment). In some embodiments, the lead 130 can include shocking elements, electrodes, conductors, etc. configured to delivery relatively low energy shock treatment to the heart H (e.g., for anti-tachycardie pacing). In some embodiments, the lead 130 can include any suitable number or combination of high energy shocking elements and/or low energy shocking elements. Moreover, the lead 130 can be delivered into the substernal space such that the shocking elements are in a desired position(s) relative to the heart H (e.g., in contact with the pericardium, near the pericardium, spaced apart from the heart H, and/or the like).

The lead delivery system 140 is configured to deliver or aid in delivering the lead 130 to a desirable location in the patient P proximate to the heart H, as described above. In some embodiments, the lead delivery system 140 may be operated by a user such as a medical professional (e.g., surgeon, etc.). The lead delivery system 140 is configured to deliver the lead 130 to a target location in the body so that the diagnostic/treatment device 110 may deliver desirable treatment, which may include pacing and/or defibrillation. In some instances, the arrangement and/or configuration of the lead delivery system 140 can allow delivery of the lead 130 to the target location in the body consistently despite differences in anatomic structures between patients. In some instances, consistent placement also allows for the first sensor 134a and the second sensor 134b to be placed in a desirable location relative to the heart H that allows the sensors 134a and 134b to detect and/or measure cardiac signals of the heart H. In some embodiments, an imaging system can be used when the lead delivery system 140 is being used to monitor the delivery of the lead 130. Thus, in some embodiments, the components of the lead delivery system 140 can include markers (e.g. radiopaque markers) similar to the marker(s) 136 of the lead 130.

The lead delivery system 140 can include, for example, a needle, an introducer (e.g., a stylet, a wire, a stiffener, etc.), guidewire, and a sheath. As described in further detail herein, the components of the lead delivery system 140 can be used to access the substernal space of the patient P and then to deliver the lead 130 to a target location within the substernal space and/or otherwise to a target location relative to the heart H.

The needle can be any suitable shape or size that allows the needle to establish access into the body. For example, the needle can be formed from any suitable biocompatible material such as any of those described above and can include a sharpened distal tip allowing the needle to puncture or pierce the skin of the patient to access an interior region of the body. In some embodiments, the needle can be formed of a material allowing one or more portions of the needle to bend, flex, and/or otherwise transition between two or more configuration, states, positions, orientations, etc. In some embodiments, the needle can be formed from a material that has sufficient stiffness to allow the needle to puncture the patient P and access the substernal space, while at least a portion of the needle has sufficient flexibility to allow at least the portion of the needle to transition between the two or more configurations, states, positions, orientations, etc. For example, in some embodiments, the needle or at least a portion thereof can be formed of a polymer material having a desired durometer. In some embodiments, the needle or at least a portion thereof can be formed of a shape memory alloy such as nickel-titanium alloy (nitinol). Forming the needle (or at least a portion thereof) of a shape memory alloy material may allow the needle to transition between two or more configurations in response to the shape memory alloy going through a phase change (i.e., a martensite phase to an austenite phase). In some embodiments, the needle may include one or more portions that are steerable or otherwise reconfigurable.

The needle can be used to first access or form an opening (e.g., initial entry point) into the substernal space. The needle defines a needle lumen that extends through the entire length of the needle from a proximal end of the needle to a distal end of the needle. The needle and the needle lumen may be sized so that the stylet and the guidewire can fit inside the needle lumen. The needle includes a biased portion along a length of the needle. For example, the biased portion can be and/or can be positioned along the distal end portion of the needle. The biased portion can be configured to transition between, for example, a biased configuration, state, position, orientation, etc. and an unbiased configuration, state, position, orientation, etc. For example, in the biased configuration, the biased portion of the needle can be angled away from an otherwise straight central (or longitudinal) axis extending through the needle. In some embodiments, the biased portion is angled to facilitate delivery and/or placement of the lead 130 against the posterior sternal wall and up to or near, for example, the sternal angle, the angle of Louis, and/or any other suitable position (e.g., a superior position). In some embodiments, the biased portion of the needle forms a bend at an angle associated with an angle of the posterior sternal wall. In some embodiments, the biased portion can be between about 5% and about 50% of the length of the needle (or at least the distal end portion thereof). In some embodiments, the angle of the biased portion in the biased configuration can be between about 1 degree and about 45 degrees (or any suitable angle or range of angles therebetween) from the central axis of the needle lumen at the proximal end.

The introducer can be any suitable member, device, mechanism, etc. configured to facilitate the introduction or insertion of the needle into the body of the patient. For example, the introducer can be a stylet that is configured to be at least temporarily disposed in the needle lumen to selectively straighten the biased portion. In some embodiments, the stylet is substantially straight to allow the stylet to straighten the biased portion. In such embodiments, the stylet can have a sufficient stiffness to straighten or substantially straighten the biased portion of the needle. During operation, such a stylet can be disposed into the needle when needle is being inserted into the substernal space to a desired, target, and/or predefined position, then removed to allow the biased portion of the needle to transition toward (e.g., return to) the biased configuration. For example, the stiffness and/or rigidity of the stylet that acts to straighten the biased portion of the needle (e.g., to place the biased portion in an unbiased configuration) is removed when the stylet is withdrawn from the needle, thereby allowing the biased portion to transition toward and/or return to the biased configuration.

In some embodiments, the introducer can be a stylet that includes a biased portion that is configured to transition between at least a biased configuration and an unbiased configuration. For example, the stylet can be biased in an opposite direction relative to the biased portion of the needle. For example, the stylet can be inserted into the needle and oriented such that the biased portions of the needle and the stylet counteract each other so that the needle is substantially straight (e.g., for insertion into the substernal space). Once in the substernal space, the stylet can be reoriented, moved, rotated, and/or removed so that the biased portions align and/or are otherwise biased in the same or substantially the same direction, allowing each biased portion to return to the biased configuration. After placing the needle in a desired position, the stylet can be removed and/or withdrawn from the needle.

Although the introducer is described above as being a stylet that can be disposed in the lumen of the needle, in other embodiments, the introducer can be any suitable configuration. For example, the introducer can be a catheter, tube, sleeve, or the like configured to be disposed on or over at least a portion of the needle. In such embodiments, the introducer and/or at least a portion thereof can have a stiffness that can constrain the needle (e.g., maintain the needle in a first or straight configuration). In some embodiments, the introducer can have a sharpened distal tip configured to pierce tissue, while the needle has a rounded or atraumatic distal tip.

The guidewire (e.g., wire) is configured to be inserted into the needle lumen of the needle after the stylet is removed and the needle is positioned in a desirable position in the substernal space. The guidewire is configured to extend through the needle lumen and out of the distal end of the needle such that a distal end of the guidewire is positioned deeper in the substernal space than the distal end of the needle. In some implementations, the guidewire is inserted and/or advanced to a predetermined position such as, for example, the sternal angle. In some embodiments, the guidewire and/or at least the distal end thereof may be atraumatic to reduce potential damage tissue in the substernal space and/or adjacent tissue and organs. In some embodiments, the distal end of the guidewire may be blunt (e.g., rounded) to reduce the likelihood of damage to the tissue. In some embodiments, the guidewire may function the same or similar to the stylet. For example, the guidewire may include a biased portion or may be rigid or stiff enough to straighten the needle for insertion into the body and after insertion, the guidewire can be manipulated to allow the needle to bend to the biased configuration or position. Moreover, once the needle is in the biased configuration, the guidewire may be advanced distally beyond the needle (as described above). Accordingly, the guidewire may function as both the stylet and the guidewire. Once the guidewire is placed, the needle is configured to be removed from the patient, while the guidewire remains in the substernal space.

The sheath is configured to be inserted over and/or advanced along the guidewire. For example, the sheath defines a sheath lumen allowing the sheath to be disposed about or over the guidewire such that the guidewire is in the sheath lumen. With at least a portion of the guidewire disposed in the sheath lumen, the sheath can be advanced along the guidewire, allowing the guidewire to guide the sheath into a desired location in the substernal space. In some embodiments, the sheath is inserted a predetermined distance along the guidewire. In some embodiments, the sheath is inserted until a distal end of the sheath aligns or substantially aligns with the distal end of the guidewire. In some embodiments, the distal end (and/or any other suitable portion of the sheath) may include one or more radiopaque markers allowing the sheath to be visualized under imaging (e.g., fluoroscopy). In some embodiments, at least a distal end portion of sheath includes a dilator which is configured for blunt dissection of the soft tissue around the guidewire in the substernal space. Similarly stated, the dilator can be transitioned to a dilated state (e.g., inflated) in which the dilator pushes or dilates tissue in the substernal space resulting in an opening, hole, void, etc. that has a larger perimeter or circumference than would otherwise be caused by the advancement and/or presence of the sheath. In some embodiments, the dilator can be withdrawn from the sheath after the sheath is advanced into the desired position. In other embodiments, the dilator can be integrated into the sheath and after dilation and placement of the sheath, the integrated dilator can be transitioned to a non-dilated state. Once the sheath is placed, the guidewire can be removed from the sheath (e.g., withdrawn from the sheath lumen).

The sheath lumen is configured to receive and/or accept the lead 130. For example, an inner diameter of the sheath lumen can be sized to allow the lead 130 to be inserted and/or advanced therethrough. As described above, the sheath can be positioned in the body of the patient such that at least the distal end portion of the sheath is in the substernal space. Accordingly, the lead 130 can be advanced into the substernal space to a predetermined, target, and/or otherwise desired position. In some embodiments, lead 130 can be in a delivery configuration when disposed in the sheath lumen and configured to transition from the delivery configuration to a deployed or expanded configuration once released from the distal end portion of the sheath. For example, in some embodiments, the sheath and/or an inner wall defining the sheath lumen can be configured to constrain and/or straighten one or more features (e.g., coils, bends, biased portions, etc.) of the lead 130 when the lead 130 is in the sheath lumen.

Once the lead 130 is advanced through the sheath lumen and placed in a desired position within the substernal space, the sheath can be removed and/or retracted from the lead 130. Accordingly, the lead delivery system 140 can be used to deliver the lead 130 to a desired position within the substernal space and relative to the heart H.

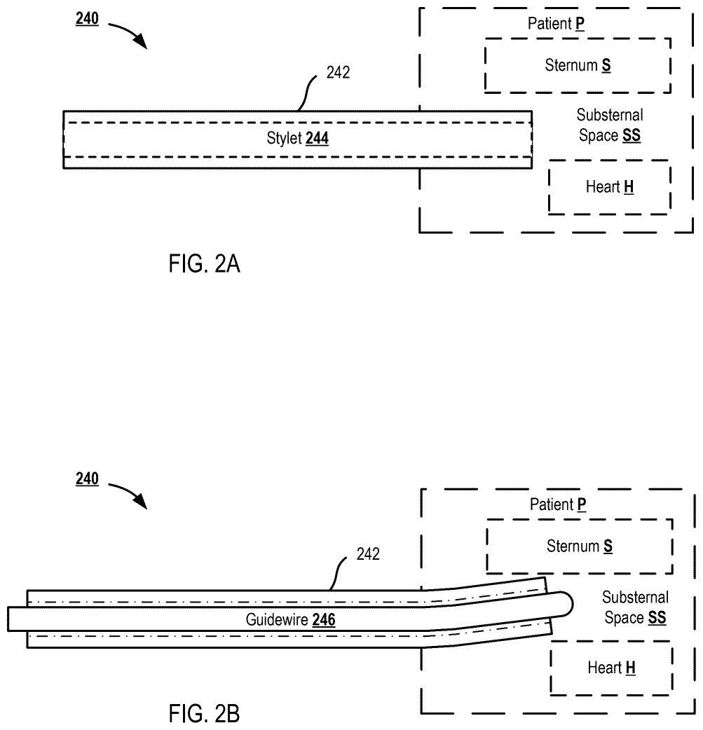

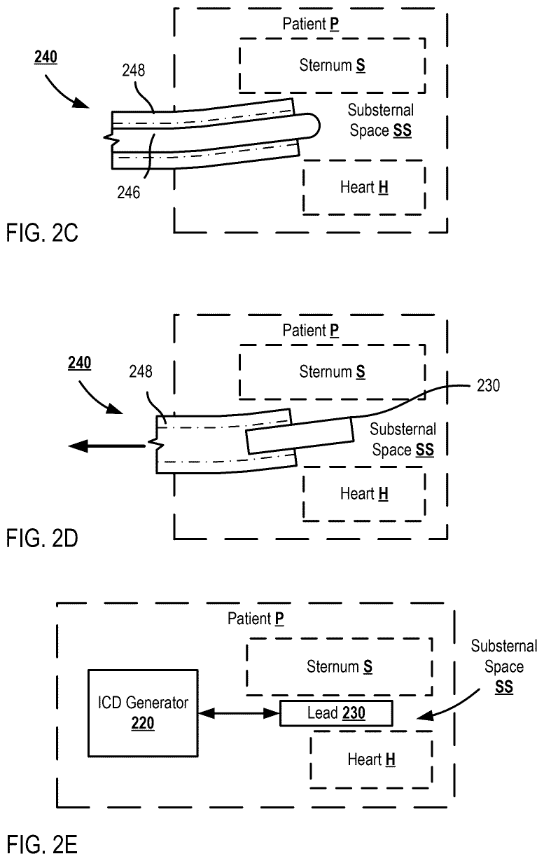

FIGS. 2A-2E schematically depict a lead delivery system 240 according to another embodiment. The lead delivery system 240 can be structurally and/or functionally similar to the lead delivery system 140 of FIG. 1. The lead delivery system 240 can be used to deliver a lead 230 of an ICD (e.g., structurally and/or functionally similar to the lead 130 of diagnostic/treatment device 110 shown in FIG. 1) into a substernal space of a patient P. For example, as described above with reference to the lead delivery system 140, the lead delivery system 240 shown in FIGS. 2A-2E includes a needle 242, a stylet 244, a guidewire 246, and a sheath 248.

FIG. 2A depicts the needle 242 having the stylet 244 disposed within a needle lumen of the needle 242. As described above with reference to the needle of the lead delivery system 140, the needle 242 includes and/or forms a biased portion configured to transition between two or more configurations (e.g., at least a biased configuration and an unbiased configuration). The stylet 244 can be configured such that when the stylet 244 is disposed in the needle lumen, the needle 242 is substantially straight. Similarly stated, the needle 242 and/or at least the biased portion thereof can be in an unbiased (e.g., straight) configuration when the stylet 244 is disposed in the needle 242.

In some embodiments, the stylet 244 is substantially straight and has a stiffness and/or rigidity that is sufficient to straighten at least the biased portion of the needle 242 when the stylet 244 is in the needle lumen. In some embodiments, the stylet 244 can include a biased portion that is configured to transition between at least a biased configuration and an unbiased configuration in a manner similar to the biased portion of the needle 242. In such embodiments, the stylet 244 can be biased in an opposite direction relative to the biased portion of the needle. As such, the stylet 244 can be oriented relative to the needle 242 such that a bias (e.g., bend) in the stylet 244 counteracts a bias (e.g., bend) in the needle 242. so that the needle is substantially straight allowing the needle 242 to be inserted into the patient and advanced toward or into a substernal space SS between the sternum S and the heart H.

Once in the substernal space SS, the stylet 244 can be reoriented, moved, rotated, retracted, and/or removed from the needle 242 to allow for a biased portion of the needle 242 to transition from the unbiased configuration (e.g., substantially straight) to or toward the biased configuration (e.g., bent, curved, angled, etc.). For example, in embodiments in which the stylet 244 is a substantially stiff and straight member, the stylet 244 can be retracted and at least partially removed from the needle 242, allowing the biased portion of the needle 242 to transition to or toward the biased configuration. In other embodiments in which the stylet 244 includes and/or forms the biased portion, the stylet 244 can be reoriented, moved, rotated and/or otherwise manipulated such that the biased portion of the stylet 244 substantially corresponds with and is in the same direction as the biased portion of the needle 242. As such, the biased portions of the needle 242 and stylet 244 collectively transition to or toward the biased configuration. As shown in FIG. 2B, the needle 242 can be biased toward the sternum S when in the biased configuration. The biased configuration of the needle 242 allows for the needle 242 to be advanced through the substernal space SS in a direction toward a posterior substernal wall, which in some implementations, may be a desirable position or a desirable area for placing the lead 230.

Once the stylet 244 is completely removed from the needle, the guidewire 246 can be inserted into the needle lumen of the needle 242. As seen in FIG. 2B, the guidewire 246 is advanced through the needle lumen, following the biased portion toward the sternum. The guidewire 246 can be advanced such that at least a distal end portion of the guidewire 246 extends further into the substernal space SS than the distal end portion of the needle 242. In some implementations, the guidewire 246 is configured to extend out of the distal end of the needle 242 and continue along the sternum S until the distal end (or distal end portion) of the guidewire 246 is in a predetermined, target, and/or desired position, such as the sternal angle and/or the like. FIG. 2B shows the distal end of the guidewire 246 having a rounded or otherwise atraumatic shape or configuration so as not to damage the sternum S, the heart H or any other tissue of the patient P. Once the guidewire 246 is positioned in the substernal space SS, the needle 242 can be removed and/or retracted along the guidewire 246 and out of the patient while the guidewire 246 remains and/or is otherwise maintained in the substernal space SS.

After the needle 242 is removed from the patient P (e.g., retracted along the guidewire 246), the sheath 248 can be advanced over the guidewire 246. For example, as shown in FIG. 2C, the sheath defines a sheath lumen that allows the sheath 248 to be advanced along the guidewire 246 into the substernal space SS to a desired location. In some embodiments, the sheath 248 is inserted a predetermined distance along the guidewire 246. In some embodiments, the sheath 248 is inserted until a distal end of the sheath 248 aligns or substantially aligns with the distal end of the guidewire 246. In some embodiments, the distal end (and/or any other suitable portion of the sheath 248) may include one or more radiopaque markers allowing the sheath 248 to be visualized under imaging (e.g., fluoroscopy). In some embodiments, at least the distal end portion of the sheath 248 includes a dilator configured to aid in dilating tissue around the guidewire 246 to allow the sheath 248 to be inserted into the substernal space SS. In some embodiments, the dilator can be withdrawn from the sheath 248 after the sheath 248 is advanced into the desired position. In other embodiments, the dilator can be integrated into the sheath 248 and after dilation and placement of the sheath 248, the integrated dilator can be transitioned to a non-dilated state. Once the sheath 248 is advanced to and/or disposed in a desired location, the guidewire 246 (and in some embodiments, the dilator) can be removed from the sheath 248, while the sheath 248 remains and/or is maintained in the substernal space SS.

After the guidewire 246 is removed from the sheath 248, the lead 230 can be inserted into the lumen of the sheath 248 and advanced into the substernal space SS, as seen in FIG. 2D. The lead 230 can be advanced into the substernal space SS through the sheath lumen until the lead 230 reaches the predetermined, target, and/or desired location. In some embodiments, the lead 230 is configured to be advanced past the distal end of the sheath 248. In some embodiments, the lead 230 can include one or more radiopaque markers that allow a doctor, surgeon, technician, etc., to determine the location and/or orientation of the lead 230 in the substernal space SS using one or more imaging techniques (e.g., fluoroscopy). Once the location and/or orientation of the lead 230 is confirmed, the sheath 248 can be removed from the from the patient P, while the lead 230 remains and/or is maintained in the substernal space SS.

As seen in FIG. 2E, once the sheath 248 is removed, the lead 230 remains in the substernal space SS and is configured to be coupled and/or electrically connected to the ICD generator 220 which is also implanted within the patient P, as described in reference to the control device 120 shown in FIG. 1. With the lead 230 delivered using the lead delivery system 240, the ICD 210 shown in FIG. 2E, including the ICD generator 220 and the lead 230, is configured to measure and/or otherwise receive cardiac signals associated with the electrical and/or mechanical functioning of the heart H, determine a treatment based on the signals, and apply a shock treatment to the heart H of the patient P while reducing inappropriate and/or undesirable shocks.

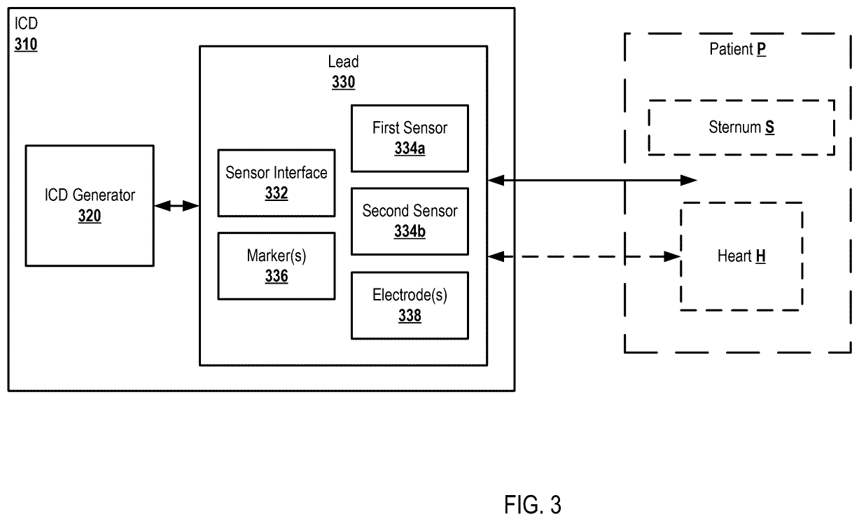

The lead delivery systems and/or methods described herein can be used to deliver any suitable lead(s) of an ICD system. For example, FIG. 3 shows an example of an ICD 310 having an ICD generator 320 and at least one lead 330 that can be delivered to a target location within a body of a patient using any of the lead delivery systems and/or methods described herein. In some embodiments, the ICD 310 can be similar to and/or substantially the same as any of the ICDs described in the '184 patent and thus, portions of the ICD 310 may not be described in detail herein. While the ICD 310 is shown as including particular components and/or features, it should be understood that the ICD 310 is presented by way of example and not limitation. Accordingly, the lead delivery systems and/or methods described herein may be used to deliver the lead 330 described below with reference to FIG. 3 (and/or any other suitable lead).

As shown in FIG. 3, the ICD 310 can include an ICD generator 320 operatively coupled to a lead 330 having a sensor interface 332, a first sensor 334a, an optional second sensor 334b, marker(s) 336, and electrode(s) 338. The ICD generator 320 is configured to determine when to provide treatment and to generate treatment energy that can be sent to the heart H via the lead 330. In some embodiments, the ICD generator 320 can be placed on the pectoralis major muscle of the patient P, behind the pectoral major muscle, on the abdomen, along the left exterior thorax, or elsewhere on or in the body of the patient P. Although not shown in FIG. 3, the ICD generator 320 can include a processor, a memory, a power system, and/or a lead interface, as described, for example, in the '184 patent.

The ICD generator 320 is electrically and/or electronically coupled to the lead 330 allowing signals and/or electric power to be transferred therebetween. For example, the ICD generator 320 can receive signals from the lead 330 that include data representing measurements associated with one or more characteristics of the patient P or the heart H. 320 The ICD generator 320 can include a power system configured to store and/or generate energy for use by the ICD 310 and to select, determine, and/or define treatments for delivery to the patient P. In some embodiments, the power system can include at least one battery (e.g., LiPo, Li-ion, etc.). The at least one battery can be charged when the battery is low on power. In some embodiments, the power system includes a primary cell battery and a rechargeable battery. In some embodiments, the power system can be configured to charge automatically (e.g., via patient P movement) or wirelessly (e.g., via inductive charging). The power system can also be configured to generate a therapy signal (e.g., anti-tachycardia pacing, defibrillator shock, etc.). For example, the therapy signal can include treatment energy for at least one of anti-tachycardia pacing and/or shock therapy. In some implementations, the treatment energy for anti-tachycardia pacing can be low-power or relatively low-power treatment energy and the treatment energy for shock therapy can be high-power or relatively high-power treatment energy. In some embodiments, the operation of the power system is controlled via a processor of the ICD generator 320 executing instructions stored in a memory of the ICD generator 320.

In some embodiments, the process of the ICD generator 320 determining the cardiac status and/or functioning of the heart can include, for example, comparing, correlating, and synchronizing the signal data received from lead 330, as described in detail in the '184 patent. For example, the signals can include cardiac electrical signals (e.g., electrocardiogram signals, cardiac electrogram signals, etc.) and cardiac mechanical signals (e.g., hemodynamic status and/or output). The ICD generator 320 can receive the signal data from the lead and can determine the cardiac status and further classify cardiac arrhythmia. Thus, determining a cardiac status and a classification of cardiac arrhythmia can include the ICD generator 320 determining whether a sensed or determined signal from the lead 330 has an expected corresponding sensed or determined second signal and/or output from the lead 330. If the signals are not what is expected, the ICD generator 320 may withhold, delay, and/or modify the ICD therapy.

The lead 330 is operatively coupled to the ICD generator 320 via the lead interface. The lead interface is configured to couple (e.g., physically couple or at least electrically couple) the ICD generator 320 to the lead 330 to allow data signals and/or electric power (e.g., for therapy) to be transferred therebetween. In some embodiments, the lead interface can be configured to format data signals and/or modulate electric power transferred between the ICD generator 320 and the lead 330 into any suitable format(s), waveform(s), energies, etc., allowing, for example, the processing of sensor data or the like by the ICD generator 320, the generation of treatment energy, and/or the application of therapy by the lead 330. The lead interface may be a flexible interface to provide flexibility for the continuous movement of the heart H and allowing the lead 330 to maintain contact with the heart H.

In some embodiments, the lead 330 can extend away from the ICD generator 320 via a conduit, connector, tube, shaft, etc., that includes a signal/power carrying wire. The lead 330 is configured to deliver treatment to the patient P and/or the heart H of the patient P. More specifically, in some implementations, the lead 330 is configured to be deployed and/or utilized at least partially outside of the heart H. Similarly stated, the lead 330 is configured for use outside of the chambers of the heart H and, as such, is not a traditional, transvenously delivered lead. For example, the lead 330 can be a pericardial lead, an epicardial lead, and/or the like (or combinations thereof).

The sensor interface 332 is configured to communicate with the sensors 334a, 334b to send and/or receive signals between the sensors 334a and 334b, and the lead 330, which in turn is in communication with the ICD generator 320. Additionally, in some embodiments, the sensor interface 332 provides the first sensor 334a and the second sensor 334b with power. In some embodiments, the sensor interface 332 can preprocess signals output by and/or received from the first sensor 334a and the second sensor 334b. The sensors 334a and/or 334b can be included and/or coupled along one or more portions of the lead 330. Alternatively, in some embodiments, the sensors 334a and 334b can be in communication with the ICD generator 320, directly or indirectly. In some embodiments, the first sensor 334a and the second sensor 334b are integrated into the lead 330. In some embodiments, at least one of the first sensor 334a and/or the second sensor 334b are remote from the lead. In some embodiments, the outputs of the sensor interface 332 and/or the first sensor 334a and/or the second sensor 334b can be calibrated based on the patient P, the position in the body of the patient P, and/or the like.

As described in detail in the '184 patent, the first sensor 334a and the second sensor 334b are configured to measure different health characteristics associated with the heart of the patient P. In some embodiments, the first sensor 334a and the second sensor 334b can measure continuously, periodically, or when a command is received by the first sensor 334a and the second sensor 334b. 334 In some embodiments, the first sensor 334a is configured to measure cardiac electrical signals and/or derivatives thereof (e.g., heart rate, voltage, P wave, QRS morphology, ST segment, T wave, electrocardiogram (ECG) signals, etc.) As described in detail above, the lead 330 can be delivered (e.g., via the lead delivery system 140 and/or 240) such that the first sensor 334a contacts, engages, and/or is otherwise in close proximity to the heart H of the patient P and/or in or on the body of the patient P such that the cardiac signals can be measured (e.g., in contact with or in close proximity to the pericardium).

In some embodiments, the second sensor 334b is a different sensor than the first sensor 334a. In some embodiments, the second sensor 334b is configured to measure cardiac mechanical signals (e.g., hemodynamic status, hemodynamic output, blood pressure or other pressures within the body, and/or derivative(s) thereof). In some embodiments, the second sensor 334b is a pressure sensor, transducer, etc. For example, the second sensor 334b may be a pressure sensor configured to measure hemodynamic pressure. In some embodiments, the sensor 334b can be configured to directly measure and/or detect hemodynamic status or a pressure associated with the hemodynamic status (e.g., blood pressure) of the heart H. In some embodiments, the second sensor 334b is a pressure sensor configured to indirectly measure and/or detect hemodynamic status or a pressure associated with the hemodynamic status. For example, as described in detail above, the lead 330 can be delivered (e.g., via the lead delivery system 140 and 240) such that the second sensor 334b is positioned in the substernal space and/or otherwise contacts, engages, or is in close proximity to the free wall of either the right, left, or both ventricles of the heart H of the patient P. In such embodiments, movement associated with the pumping/beating of the heart can result in pressure or changes in pressure in the substernal space/tissue surrounding the heart H, which in turn, can be measured and/or detected by the pressure sensor.

In some embodiments, the first sensor 334a and/or the second sensor 334b is/are located in the epicardium of the heart. In some embodiments, the first sensor 334a and/or the second sensor 334b is/are located within the heart H. For example, the first sensor 334a and/or the second sensor 334b may be located in the right heart. In some embodiments, the first sensor 334a and the second sensor 334b can be collocated or substantially collocated. In some embodiments, the first sensor 334a and/or the second sensor 334b can be remotely or separately located. For example, in some embodiments, the first sensor 334a can be positioned in contact with or adjacent to the fibrous pericardium of the heart H, while the second sensor 334b can be positioned apart from the heart H in the substernal space. In some embodiments, the first sensor 334a and/or the second sensor 334b measures characteristics of the heart H and/or other portions or body parts of the patient P.

As described in further detail herein, the sensors 334a and 334b can measure, detect, and/or sense one or more characteristics associated with the heart H and can send data associated with those characteristics to the ICD generator 320 (e.g., directly or indirectly via the lead 330). Upon receipt, the ICD generator 320 can analyze, process, aggregate, correlate, etc. the data to determine, for example, a cardiac status of the heart H. Furthermore, based on the determined and/or defined cardiac status, the ICD generator 320 can detect and/or determine the occurrence of health events, such as arrhythmia, tachycardia, and/or the like. In some embodiments, the use of data from each of the first sensor 334a and the second sensor 334b can allow the ICD generator 320 to determine the cardiac status of the heart H with greater sensitivity and specificity than when determining cardiac status using cardiac signals, QRS complex morphology, and/or other cardiac electrical signal measurements alone (e.g., determined based on signals from the first sensor 334a), hemodynamic status measurements alone (e.g., determined based on signal from the second sensor 334b), and/or other cardiac characteristics individually.

While the lead 330 is described herein as including “the first sensor 334a” and “the second sensor 334b,” it should be understood that the first sensor 334a can be a single sensing device or multiple sensing devices that collectively function as the first sensor 334a, and similarly, the second sensor 334b can be a single sensing device or multiple sensing devices that collectively function as the second sensor 334b. In some embodiments, multiple sensing devices can allow for sensor data that includes multiple signal vectors (e.g., multiple cardiac electrical and/or mechanical signal vectors). Similarly, while the system 100 is described herein as including “the first sensor 334a” and “the second sensor 334b,” it should be understood that the system 100 can include any number of additional sensors configured to sense and/or detect any suitable characteristic(s) associated with the patient (e.g., cardiac electrical and/or mechanical signals or any suitable non-cardiac signals). In some embodiments, each sensor can be configured to sense and/or detect a different characteristic associated with the heart, or more generally, the patient. In some embodiments, one or more sensors can be configured to sense or detect the same characteristic, thereby allowing for confirmation/verification of signal data and/or a desired degree of sensitivity and/or specificity in interpreting the signal data.

The marker(s) 336 (e.g., radiopaque markers, landmarks, etc.) is/are one or more markers that are visible during imaging (e.g., fluoroscopy, etc.) by an imaging device when the lead 330 is disposed in the body. In some embodiments, the marker(s) 336 are radiopaque. The marker(s) 336 can allow for the position and/or orientation of the lead 330 during and after delivery to be confirmed. For example, one or more portions of the lead 330 having certain features and/or elements can include a marker 336 allowing visualization of the feature(s) and/or element(s) within the body via imaging such as fluoroscopy or the like. In some embodiments, for example, the lead 330 can include one or more markers 336 corresponding to, co-located with, and/or otherwise associated with the electrodes 338, the first sensor 334a, and/or the second sensor 334b, which can aid in positioning and/or orienting the lead 330 or confirming the position and/or orientation of the lead 330 relative to the heart H. In some embodiments, the lead 330 can also include one or more markers 336 corresponding to any suitable position and/or feature of the lead 330 (e.g., a distal end of the lead 330, electrode or sensor regions, physical features or geometries, etc.).

As shown in FIG. 3, the lead 330 includes one or more electrodes, shocking elements, conductors, etc. (referred to herein as electrodes 338) for delivering treatment (e.g., including energy from the generator 320) to the heart H of the patient P. In some embodiments, the lead 330 can include biased portions, loops, coils, waves, and/or any other shape, geometry, and/or feature that can aid in positioning all or a subset of the electrodes 338 relative to the heart H when the lead 330 is in the substernal space. In some embodiments, the lead 330 is positioned in contact with, adjacent to, and/or otherwise in close proximity to the fibrous pericardium of the heart H. In some embodiments, the lead 330 can be designed and/or formed to use, traverse, and/or fill (or at least substantially use, traverse, and/or fill) at least a portion of the volume between the sternum and the pericardium of the heart H, thereby allowing a first portion of the lead 330 to be in contact with a posterior sternal wall and a second portion of the lead 330, including one or more electrodes 338, to be in contact with, or in close proximity to, the pericardium of the heart H. For example, in some embodiments, the electrodes 338 positioned in contact with or close to the pericardium may be lower energy shock producing elements (e.g., pacing electrodes). In some embodiments, a portion of the electrodes 338 can be spaced apart from the heart H (e.g., in contact with the posterior sternal wall and/or otherwise in the substernal space). In such embodiments, these electrodes 338 may be, for example, higher energy shock producing elements (e.g., a coil capable of delivering defibrillation treatment). In such embodiments, the energy delivered by the higher energy shock elements can be sufficiently high that the shocking elements can be spaced apart from the heart H while still being able to provide the desired shock therapy (defibrillation).

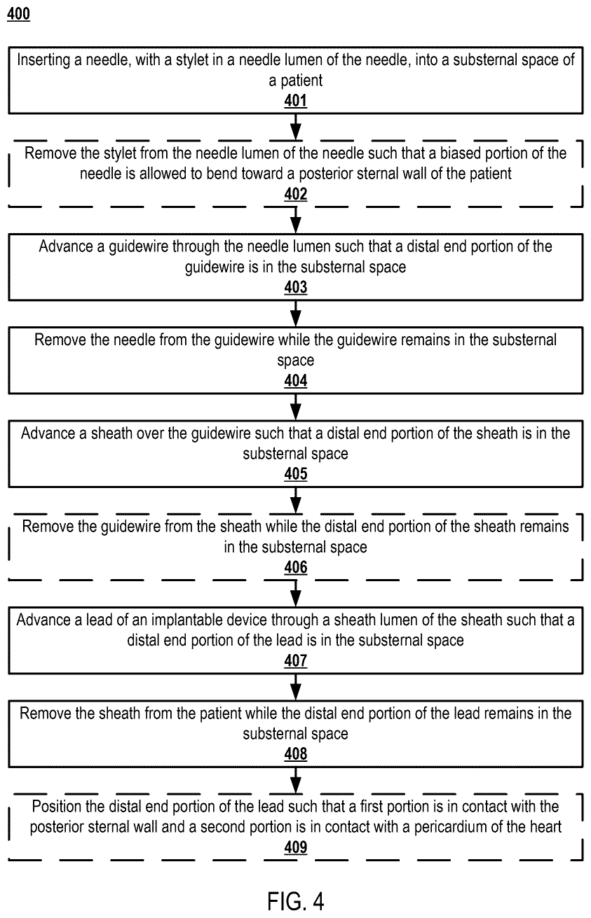

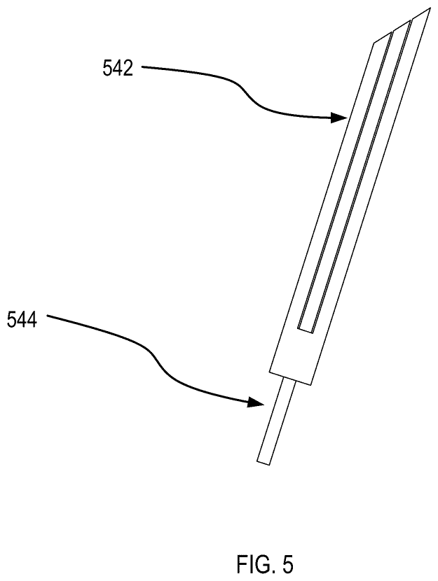

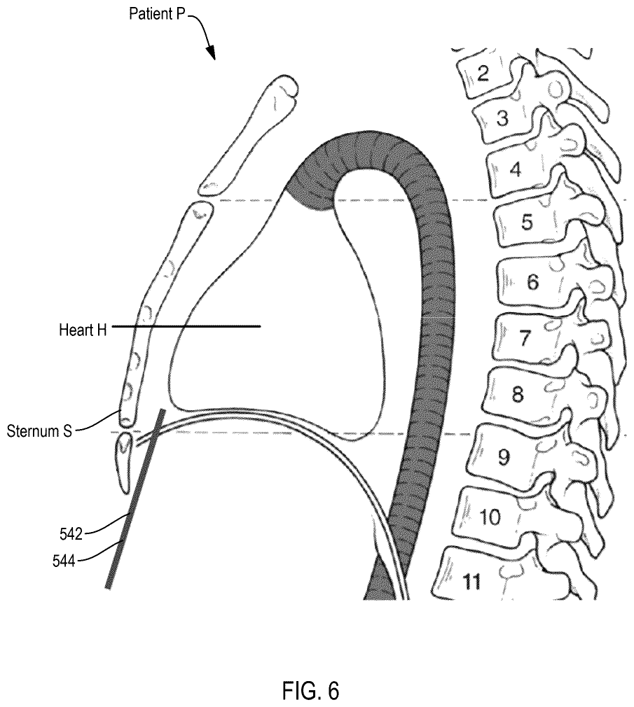

FIG. 4 is a flow chart depicting a method 400 for positioning a lead (e.g., functionally and/or structurally similar to any of the leads 130, 230, and/or 330) in the substernal space of a patient, according to an embodiment. In some embodiments, the method 400 can be completed by, for, and/or with the system 100 and/or 200. For example, the method 400 can be used to position and/or deliver into the substernal space a lead for a diagnostic/treatment device or system such as an ICD and/or the like. The method 400 allows for the lead to be delivered to a predetermined, target, and/or desired position in the substernal space by a lead delivery system (e.g., functionally and/or structurally similar to the lead delivery system 140 of FIG. 1 and/or the lead delivery system 240 of FIGS. 2A-2D) so that the diagnostic/treatment device can, for example, deliver treatment as desired. Desirable placement of the lead in the substernal space allows for accurate sensing and determination of whether a health event is occurring which allows for the diagnostic treatment device to treat more accurately (e.g., with greater sensitivity and specificity) the health event and reduce the likelihood of sudden cardiac arrest. In some instances, the method 400 may be used to deliver a lead, which in turn, may allow for improved treatment for ventricular tachycardia and/or ventricular fibrillation, while reducing the delivery of inappropriate and/or undesirable shocks.

At 401, the method 400 includes inserting a needle (e.g., structurally and/or functionally similar to the needle 242 of FIGS. 2A-2B), with a stylet (e.g., structurally and/or functionally similar to the stylet 244 of FIGS. 2A) in a needle lumen, into a substernal space of a patient. The stylet is configured to be disposed in the needle lumen and to substantially straighten a biased portion of the needle, as described in detail above with reference to specific embodiments. The needle is inserted into the substernal space from below the sternum. In some embodiments, the step at 401 is similar to and/or otherwise schematically represented by the embodiment shown in FIG. 2A.

At 402, the method 400 optionally includes removing the stylet from the needle lumen of the needle such that a biased portion of the needle is allowed to transition or bend toward a posterior sternal wall of the patient. In some embodiments, the stylet is reoriented within the needle lumen to allow the needle to bend toward the posterior sternal wall. For example, the stylet can include a biased portion that, when in an orientation opposite the needle bias, straightens the needle and, when in an orientation aligned with the needle, bends with the needle. In some embodiments, the needle does not include a bias and is substantially straight or includes a bias that is not affected by the stylet. In some implementations, allowing and/or configuring the needle to bend toward the posterior sternal wall allows for desirable positioning of the lead.

At 403, the method 400 includes advancing a guidewire (e.g., structurally and/or functionally similar to the guidewire 246 of FIGS. 2B-2C) through the needle lumen such that a distal end portion of the guidewire is in the substernal space. In some embodiments, the guidewire is extended to a position at or in close proximity to the sternal angle. In some embodiments, the guidewire follows the needle bias as the guidewire is advanced distally, allowing the guidewire to be moved toward and/or along the posterior wall of the sternum. In some embodiments, the step at 403 is similar to and/or otherwise schematically represented by the embodiment shown in FIG. 2B. At 404, the method 400 includes removing the needle from the guidewire while the guidewire remains in the substernal space.

At 405, the method 400 includes advancing a sheath (e.g., structurally and/or functionally similar to the sheath 248 of FIGS. 2C-2D) over the guidewire such that a distal end portion of the sheath is in the substernal space. In some embodiments, the sheath is inserted a predetermined distance along the guidewire. In some embodiments, the sheath is inserted to a predetermined position in the substernal space. In some embodiments, the sheath is inserted such that a distal end of the sheath is near a distal end of the guidewire. In some embodiments, at least the distal end portion of the sheath includes a dilator that can be used to dilate the tissue around the guidewire to allow the sheath to be advanced into the substernal space. In some embodiments, the step at 405 is similar to and/or otherwise schematically represented by the embodiment shown in FIG. 2C. At 406, the method 400 optionally includes removing the guidewire from the sheath while at least the distal end portion of the sheath remains in the substernal space. In some embodiments, 406 also optionally includes removing the dilator form the sheath. In other embodiments, the dilator may be integrated into the sheath.

At 407, the method 400 includes advancing the lead through a sheath lumen of the sheath such that at least a distal end portion of the lead is in the substernal space. In some embodiments, the distal end portion of the lead is advanced so that a distal end extends past the distal end of the sheath. In some embodiments, the lead can include one or more radiopaque markers and/or the like allowing the position of the lead to be confirmed using any suitable imaging technique. In some embodiments, the step at 407 is similar to and/or otherwise schematically represented by the embodiment shown in FIG. 2D.

At 408, the method 400 includes removing the sheath from the patient while at least the distal end portion of the lead remains in the substernal space. In some embodiments, the step at 408 is similar to and/or otherwise schematically represented by the embodiment shown in FIG. 2E. During withdrawal of the sheath, in some embodiments, the position of the lead (and/or sheath) can be monitored and/or confirmed by an imaging device showing one or more radiopaque markers on the lead (and/or sheath). At 409, the method optionally includes positioning the distal end portion of the lead such that a first portion of the lead is in contact with the posterior sternal wall and a second portion of the lead is in contact with a fibrous pericardium of the heart. In some embodiments, the positioning the distal end portion of the lead can be confirmed using the imaging techniques described above. In some embodiments, a first radiopaque marker can be associated with a position of the first portion of the lead and a second radiopaque marker can be associated with a position of the second portion of the lead. In some embodiments, the first portion corresponds to and/or may otherwise include, for example, one or more high power shocking element and the second portion corresponds to and/or may otherwise include, for example, one or more low power shocking element. In some embodiments, the step at 409 can also include positioning sensors of the lead into desired locations relative to the heart. Once the lead is positioned in a desirable location and orientation, the lead can be used in or with a diagnostic/treatment device to, for example, deliver treatment, generated by a generator, to the heart of the patient, as described in detail above.

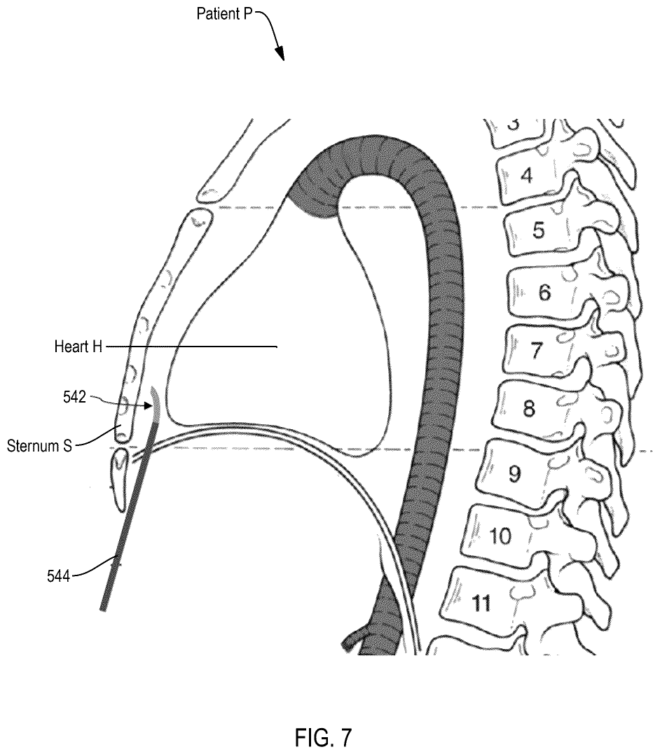

Referring generally to FIGS. 5-12, one or more portions of a lead delivery system (e.g., functionally and/or structurally similar to the lead delivery system 140 of FIG. 1 and/or the lead delivery system 240 of FIGS. 2A-2D) is/are shown in operation in the chest of a patient P. The lead delivery system shown in FIGS. 5-12 is used to access a substernal space between the heart H and the sternum S of a patient P, as described in detail above. The lead delivery system is configured to decrease the likelihood of damaging tissue in and around the substernal space as well as positioning a lead (e.g., structurally and/or functionally similar to the lead 130 of FIG. 1 and/or the lead 230 of FIG. 2E) in the substernal space. Similar to other lead delivery systems described herein, the lead delivery system shown in FIGS. 5-12 includes a needle 542 (e.g., functionally and/or structurally similar to the needle 242 of FIGS. 2A-2B), a stylet 544 (e.g., functionally and/or structurally similar to the stylet 244 of FIG. 2A), a guidewire 546 (e.g., functionally and/or structurally similar to the guidewire 246 of FIG. 2B-2C), and a sheath 548 (e.g., functionally and/or structurally similar to the sheath 248 of FIG. 2C-2D). The lead delivered using the lead delivery system shown in FIGS. 5-12 can be a lead of a diagnostic/treatment device such as an ICD and/or the like.

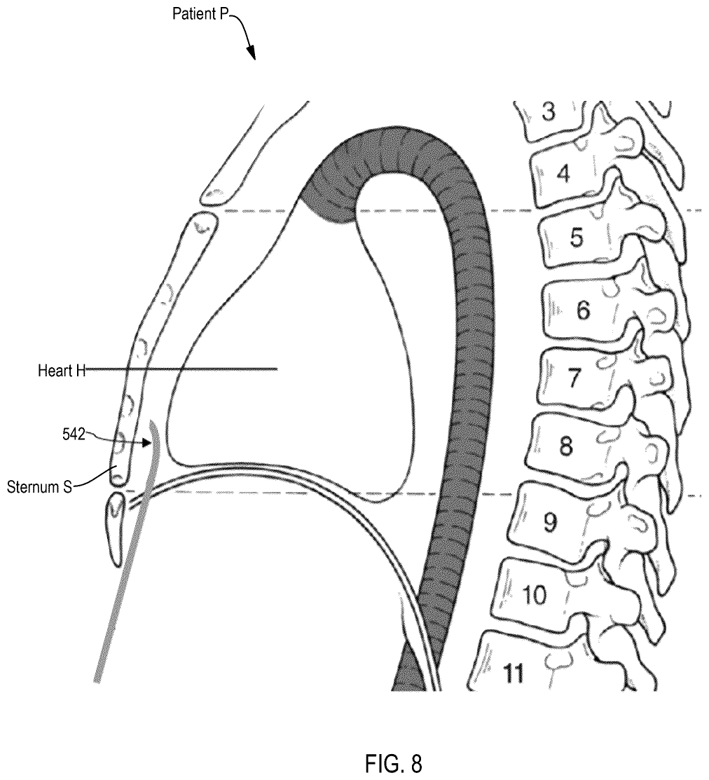

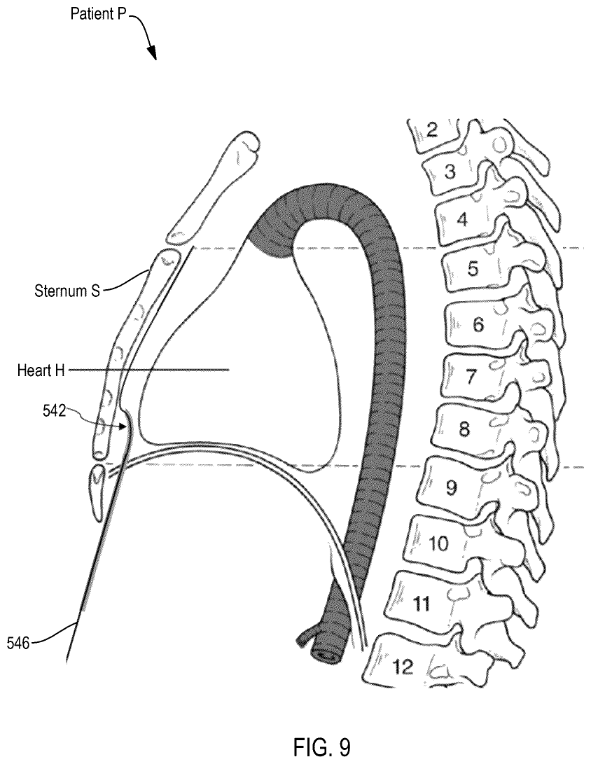

FIG. 5 depicts a stylet 544 disposed within a needle 542, according to an embodiment. In some embodiments, the stylet 544 is inserted into a proximal end of the needle 542 outside of the body of the patient. The stylet 544 is disposed within a lumen of the needle 542. In some embodiments, the stylet 544 is configured to straighten a biased portion of the needle 542, as described in detail above with reference to the lead delivery system 140 and/or 240. FIG. 6 depicts the needle 542 with the stylet 544 disposed in the needle lumen inserted in the patient and advanced to or toward a substernal space of the patient. As seen in FIG. 6, the stylet 544 straightening the needle 542 allows for the needle to enter the space between the sternum S and the heart H. As seen in FIG. 7, needle 542 can be further pushed into the substernal space toward the sternum S and away from the heart H when the stylet 544 is at least partially retracted. In other embodiments, the stylet 544 can be manipulated and/or reoriented such that the needle 542 and stylet 544 collectively transition to or toward a biased configuration, as described in detail above with reference to the lead delivery system 140 and/or 240. As seen in FIG. 7, the biased portion of the needle 542 curves away from the heart H and toward a posterior sternal wall of the sternum S. FIG. 8 depicts the needle 542 with the stylet 544 removed from the patient P so that the needle lumen of the needle 542 is empty or devoid of components and/or structures. As seen in FIG. 8, the needle 542 includes a substantially straight portion at or along the proximal end portion of the needle 542 and the biased portion at or along the distal end portion of the needle 542.

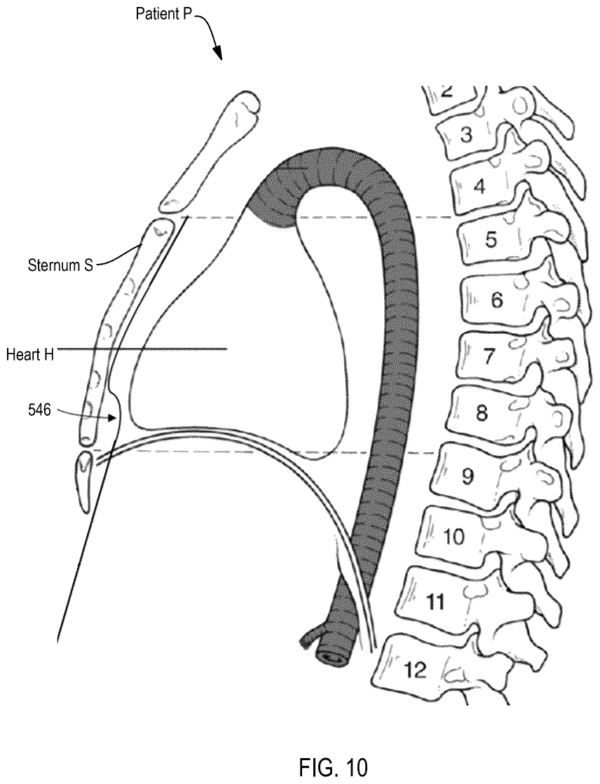

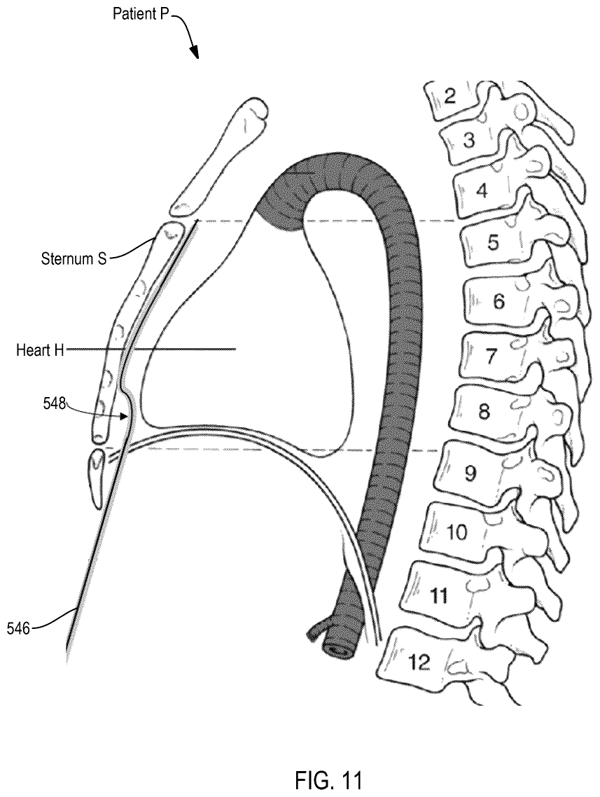

FIG. 9 depicts the needle 542 with the guidewire 546 disposed within and extending through the needle lumen and into the substernal space. The bias of the needle 542 allows for the guidewire 546 to be advanced along the posterior sternal wall and away from the heart H, as shown in FIG. 9. The guidewire 546 provides a path along which the sheath 548 can be advanced, which in turn, aids in avoiding damaging the heart H during an ICD delivery procedure. In some embodiments, the guidewire 546 is advanced to a predetermined, target, and/or desired position. In some embodiments, such as the embodiment shown in FIG. 9, the predetermined position is the sternal angle, but any other position along the posterior sternal wall may be suitable. Once the guidewire 546 is in the desired position, the needle 542 is retracted and withdrawn from the body of the patient P, while the guidewire 546 remains and/or is maintained in the desired position, as depicted in FIG. 10.

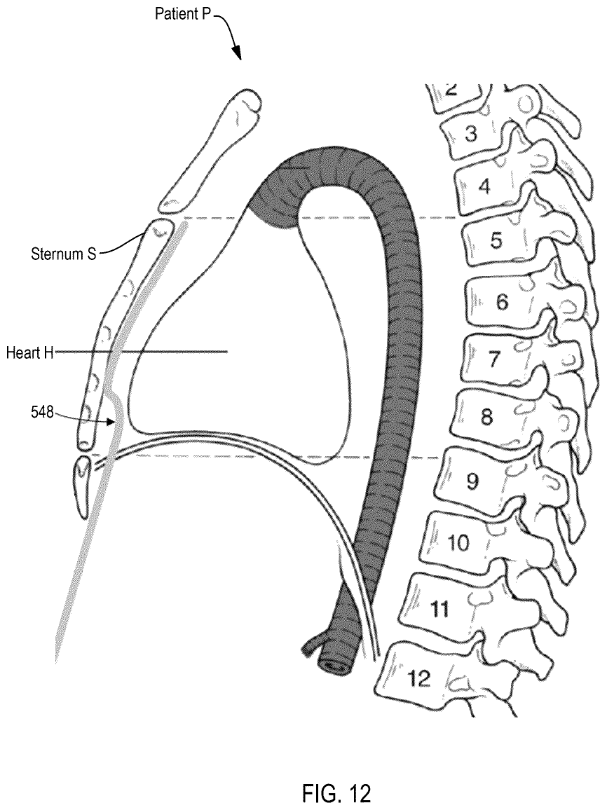

FIG. 11 depicts the sheath 548 advanced along and disposed on the guidewire 546. The sheath 548 defines a sheath lumen allowing the sheath 548 to be disposed over and/or about the guidewire 546 and advanced distally to the distal end portion of the guidewire 546. In some embodiments, one or more portions of the sheath 548 include(s) a dilator. For example, the distal end portion of the sheath 548 may be coupled to and/or may include a dilator. The dilator is configured to dilate the tissues around the guidewire 546 as the sheath 548 is advanced over the guidewire 546, thereby allowing the sheath 548 to be placed in a desired position within the substernal space. In some embodiments, the dilator can be removed after the sheath 548 is advanced to the desired position. In other embodiments, the dilator can be integrated with a portion of the sheath 548 (e.g., the distal end portion) and can be transitioned to a non-dilated state when the sheath 548 is advanced to the desired position. In some embodiments, the desired position is at and/or near the distal end or distal end portion of the guidewire 546. In some embodiments, the desired position is the sternal angle and/or any other anatomic structure or landmark. Once the sheath 548 is in the desired position, the guidewire 546 is retracted through the sheath lumen and withdrawn from the body of the patient P, while the sheath 548 remains and/or is maintained in the desired position, as depicted in FIG. 12. In some embodiments, the dilator is also removed from the sheath 548 in the same, parallel, and/or independent process as the removal of the guidewire 546.

The sheath 548 with the guidewire 546 removed is configured to receive and/or accept a lead so that the lead can be placed in a desired location in the substernal space. As described in detail above, the desired location of the lead may allow one or more portions, features, and/or components of the lead (e.g., sensor(s), electrode(s), and/or the like) to be in contact with and/or in close proximity to the fibrous pericardium. In addition, the desired location of the lead may allow one or more other portions, features, and/or components of the lead (e.g., other electrode(s), structural supports, anchors, etc.) to be in contact with and/or in close proximity to the posterior sternal wall. In some embodiments, delivering the lead in such a manner and/or to such a location can allow the lead to be in a stable or relatively stable position within the substernal space, thereby allowing the ICD to provide shock therapy while reducing inappropriate and/or undesirable shocks.

While various schematics, embodiments, and/or implementations have been described above, it should be understood that they have been presented by way of example only, and not limitation. Various modifications, changes, and/or variations in form and/or detail may be made without departing from the scope of the disclosure and/or without altering the function and/or advantages thereof unless expressly stated otherwise. Likewise, while embodiments and/or features, components, configurations, aspects, etc. thereof may be described above in the context of certain implementations, it should be understood that such implementations are presented by way of example only and not limitation. Any of the embodiments and/or features, components, configurations, aspects, etc. thereof can be used in, and/or adapted for use in, other implementations unless expressly stated otherwise. Functionally equivalent embodiments, implementations, and/or methods, in addition to those described herein, will be apparent to those skilled in the art from the foregoing descriptions and are intended to fall within the scope of the disclosure.

Where schematics, embodiments, and/or implementations described above indicate certain components arranged in certain orientations, configurations, or positions, the arrangement of components may be modified. Although various embodiments have been described as having particular features, configurations, and/or combinations of components, other embodiments are possible having a combination of any features, configurations, and/or components from any of embodiments described herein, except mutually exclusive combinations. The embodiments described herein can include various combinations and/or sub-combinations of the functions, components, configurations, and/or features of the different embodiments described.

The specific configurations of the various components can also be varied. For example, the size and specific shape of the various components can be different from the embodiments shown, while still providing the functions as described herein. More specifically, the size and shape of the various components can be specifically selected for a desired or intended usage. Thus, it should be understood that the size, shape, and/or arrangement of the embodiments and/or components thereof can be adapted for a given use unless the context explicitly states otherwise.

Where methods described above indicate certain events occurring in certain order, the ordering of certain events may be modified. Additionally, certain of the events may be performed concurrently in a parallel process, when possible, as well as performed sequentially as described above. While methods have been described as having particular steps and/or combinations of steps, other methods are possible having a combination of any steps from any of methods described herein, except mutually exclusive combinations and/or unless the context clearly states otherwise.

What is claimed is:

1. A system for delivering a lead of an implantable diagnostic/treatment device, the system comprising:
a needle defining a needle lumen, the needle configured to be transitioned between a first configuration in which the needle is straight for insertion into a substernal space of a patient and a second configuration in which at least a distal end portion of the needle is biased toward a posterior sternal wall when disposed in the substernal space of the patient;
an introducer configured to selectively maintain the needle in the first configuration during insertion into the substernal space, the introducer configured to be removed from the needle after insertion to allow the needle to transition to the second configuration;
a guidewire configured to extend through the needle lumen and into the substernal space of the patient; and
a sheath defining a sheath lumen, the sheath configured to be advanced over the guidewire to dispose a distal end of the sheath in a desired position in the substernal space, the sheath lumen allowing the lead to be advanced therethrough to deliver the lead to the substernal space such that at least a portion of the lead is in contact with the posterior sternal wall.

2. The system of claim 1, wherein the introducer is a stylet removably disposed in the needle lumen.

3. The system of claim 2, wherein the introducer is configured to removably engage the needle to straighten a bend along a length of the needle.

4. The system of claim 1, wherein the needle in the second configuration is biased at an angle associated with an angle of the posterior sternal wall.

5. The system of claim 1, wherein a distal end portion of the sheath includes a dilator.

6. The system of claim 1, wherein a distal end of the needle is atraumatic.

7. The system of claim 1, wherein a distal tip of the guidewire is atraumatic.

8. A system, comprising:
an implantable cardioverter defibrillator (ICD) configured to be implanted in a patient, the ICD including a generator configured to generate treatment energy and a lead configured to deliver the treatment energy from the generator to a heart of the patient, the lead including at least one sensor; and
a lead delivery system configured to deliver the lead to a predefined location in a substernal space of a patient, the lead delivery system including:
a needle defining a needle lumen, the needle configured to transition from a first configuration in which the needle is straight for insertion into the substernal space to a second configuration such that when the needle is in the substernal space, at least a portion of the needle is biased toward the predefined location;
a guidewire configured to extend through the needle lumen and into the substernal space; and
a sheath defining a sheath lumen with a dilator, the sheath configured to be advanced over the guidewire to dispose a distal end of the sheath in the substernal space, the sheath lumen configured to accept the lead.

9. The system of claim 8, wherein the lead delivery system further includes:
an introducer configured to removably engage the needle to maintain the needle in the first configuration during insertion of the needle into the substernal space.

10. The system of claim 9, wherein the introducer is a substantially straight stylet configured to straighten the portion of the needle when disposed in the needle lumen.

11. The system of claim 8, wherein the lead includes a first sensor configured to measure cardiac electrical signals and a second sensor configured to measure cardiac mechanical signals.

12. The system of claim 11, wherein the first sensor is configured to measure at least one of electrocardiogram signals or cardiac electrogram signals radiating from a heart of the patient.

13. The system of claim 8, wherein the lead delivery system is configured to deliver the lead to the substernal space such that a portion of the lead is in contact with a posterior surface of a sternum of the patient.

14. The system of claim 8, wherein a distal tip of the guidewire is atraumatic.

15. The system of claim 8, wherein a distal end of the needle is atraumatic.

16. The system of claim 8, wherein the lead includes radiopaque markers configured to be visible to an imaging device.

17. A method for delivering a lead of an implantable device, the method comprising:
inserting a needle into a substernal space of a patient, the needle being in a first configuration during inserting;
transitioning the needle from the first configuration to a second configuration such that at least a distal end portion of the needle is biased at an angle corresponding to a posterior sternal wall of the patient;
advancing, while the needle is in the second configuration, a guidewire through a needle lumen of the needle such that a distal end portion of the guidewire is disposed in the substernal space;
removing the needle from the guidewire while the guidewire remains in the substernal space;
advancing a sheath over the guidewire such that a distal end portion of the sheath is disposed in the substernal space;
advancing the lead through a sheath lumen of the sheath such that at least a portion of the lead is disposed in the substernal space and in contact with the posterior sternal wall; and
removing the sheath from the patient while at least the portion of the lead remains in the substernal space and in contact with the posterior sternal wall.

18. The method of claim 17, further comprising:
removing the guidewire from the sheath, prior to advancing the lead, while the distal end portion of the sheath remains in the substernal space.

19. The method of claim 17, wherein the sheath includes a dilator, the method further comprising:
withdrawing the dilator after advancing the sheath over the guidewire and prior to advancing the lead through the sheath lumen.

20. The method of claim 17, wherein:
the inserting the needle into the substernal space includes inserting the needle while an introducer selectively engages the needle to maintain the needle in the first configuration; and
the transitioning the needle includes manipulating the introducer to allow the needle to transition from the first configuration to the second configuration.

21. The method of claim 20, wherein the introducer is a stylet configured to straighten at least a portion of the needle when disposed in a needle lumen, the transitioning the needle from the first configuration to the second configuration includes at least one of withdrawing the stylet from the needle lumen or rotating the stylet while the stylet is in the needle lumen to facilitate a biasing of at least the distal end portion of the needle toward the posterior sternal wall.

22. The method of claim 17, wherein the advancing the lead through the sheath lumen includes positioning the lead such that a first portion of the lead is disposed in the substernal space and in contact with the posterior sternal wall and a second portion of the lead is disposed in the substernal space and in contact with a pericardium of a heart of the patient.

23. The method of claim 22, wherein the implantable device is an implantable cardioverter defibrillator, the first portion of the lead includes at least one coil capable of delivering high voltage shocks to defibrillate the heart of the patient, and the second portion of the lead includes at least one pacing electrode configured to deliver low voltage shocks to pace the heart of the patient.

24. A method for delivering a lead of an implantable device into a patient, the method comprising:
inserting a needle in a first configuration into a substernal space of the patient;
transitioning the needle from the first configuration to a second configuration such that at least a distal end portion of the needle is biased at an angle corresponding to a posterior sternal wall of the patient;
advancing, over a guidewire delivered via a needle lumen of the needle, a sheath to place a distal end portion of the sheath in a desired position relative to the posterior sternal wall of the patient;
advancing the lead through a sheath lumen of the sheath to place at least a portion of the lead in the substernal space; and
withdrawing the sheath from the patient while at least the portion of the lead remains in the substernal space such that a first portion of the lead is in contact with the posterior sternal wall and a second portion of the lead is in contact with a pericardium of a heart of the patient.

25. The method of claim 24, wherein the implantable device is an implantable cardioverter defibrillator, the first portion of the lead including at least one coil configured to deliver high voltage shocks to defibrillate the heart of the patient and the second portion of the lead including at least one pacing electrode configured to deliver low voltage shocks to pace the heart of the patient.

26. The method of claim 24, wherein the sheath includes a dilator, the method further comprising:
dilating at least a portion of tissue in the substernal space; and
withdrawing the dilator after advancing the sheath over the guidewire and prior to advancing the lead through the sheath lumen.

27. The method of claim 24, wherein:
the inserting the needle into the substernal space includes inserting the needle while an introducer selectively engages the needle to maintain the needle in the first configuration; and
the transitioning the needle includes manipulating the introducer to allow the needle to transition from the first configuration to the second configuration.

28. The method of claim 27, wherein the introducer is a stylet configured to straighten at least a portion of the needle when disposed in the needle lumen, the transitioning the needle from the first configuration to the second configuration includes at least one of withdrawing of the stylet from the needle lumen or rotating the stylet within the needle lumen to facilitate at least the distal end portion of the needle biasing toward the posterior sternal wall.

29. The method of claim 28, wherein the transitioning the needle from the first configuration to the second configuration includes withdrawing of the stylet from the needle lumen, the method further comprising:
advancing, after withdrawing the stylet, the guidewire through the needle lumen to place a distal end portion of the guidewire in a desired position relative to the posterior sternal wall.

30. The method of claim 29, further comprising:
removing the guidewire from the sheath lumen, prior to advancing the lead, while the distal end portion of the sheath remains in the substernal space.
